(19) United States
(12) Patent Application Publication
(10) Pub. No.: US 2013/0123550 A1
(43) Pub. Date: May 16, 2013

(54) COPPER HYDROGENATION CATALYST, ESPECIALLY FOR CONVERTING OXALATE TO ETHYLENE GLYCOL, METHOD OF PREPARING THE CATALYST AND APPLICATIONS THEREOF

(71) Applicant: Tianjin University, Tianjin (CN)

(72) Inventors: Xinbin MA, Tianjin (CN); Yujun ZHAO, Tianjin (CN); Shengping WANG, Tianjin (CN); Jing LV, Tianjin (CN); Baowei WANG, Tianjin (CN); Zhenhua LI, Tianjin (CN); Yan XU, Tianjin (CN)

(73) Assignee: Tianjin University, Tianjin (CN)

(21) Appl. No.: 13/723,221

(22) Filed: Dec. 21, 2012

Related U.S. Application Data
(63) Continuation-in-part of application No. PCT/CN2011/076038, filed on Jun. 21, 2011.

(30) Foreign Application Priority Data
Jun. 24, 2010 (CN) 201010207694.8

(51) Int. Cl. C07C 29/149; B01J 23/80; B01J 23/83; B01J 23/72; B01J 23/889

(52) U.S. Cl. CPC C07C 29/149; B01J 23/72; B01J 23/8892; B01J 23/83; B01J 23/80
USPC 568/864; 502/244; 502/345; 502/242; 502/241

(57) ABSTRACT

A copper catalyst for producing ethylene glycol by hydrogenation of an oxalate. The catalyst includes a carrier, an additive, and an active component. The carrier is ceramic or metallic honeycomb. The additive is Al, Si, Ba, Ca, Ti, Zr, Fe, Zn, Mn, V, La, Ce, an oxide thereof, or a mixture thereof. The active component is copper, and the active component and the additive are coated on the carrier to form a coating layer. The additive accounts for 5-90 wt. % of the carrier, the active component accounts for 1-40 wt. % of the carrier, and the copper accounts for 5-50 wt. % of the coating layer.

### CROSS-REFERENCE TO RELATED APPLICATIONS

This application is a continuation-in-part of International Patent Application No. PCT/CN2011/076038 with an international filing date of Jun. 21 2011, designating the United States, now pending, and further claims priority benefits to Chinese Patent Application No. 201010207694.8 filed Jun. 24, 2010. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 14781 Memorial Drive, Suite 1319, Houston, Tex. 77079.

### FIELD OF THE INVENTION

The invention relates to a monolithic structure catalyst for producing ethylene glycol by hydrogenation of an oxalate, a preparation method thereof, and the application of the catalyst for producing ethylene glycol by hydrogenation of the oxalate in a fixed bed reactor.

### BACKGROUND OF THE INVENTION

Ethylene glycol (EG) is an important basic organic material. During the World War I, ethylene nitrate was used as substitute of glycerin to produce explosive because it could lower the freezing point of glycerin. Soon afterward, several feasible processes were developed. Hydrogenation of dichloromethane was used in Germany; chlorohydrin was used as raw stuff in America for the rapid growth of application of antifreeze with the development of automobile industry in the 1920s; ethylene and ethylene oxide was introduced as material which accelerated the process since polyester was developed.

The process of ethylene oxide hydration has several defects, such as longer process, high mole ratio of water and ethylene oxide, large energy consumption and low selectivity of EG. With the exhausting of oil around the world, experts are paying close attention to the processes without oil especially to the ones that using cheap resource as raw stuff. The process using syngas as material is mainly divided into two categories: direct synthesis and indirect synthesis. The promising method mainly contains formaldehyde dimerization, formaldehyde electrochemical hydrogenation dimer method, formaldehyde hydrogen formylation, glycolic acid method, formaldehyde condensation method, oxalate method and etc. The process of oxalate hydrogenation has several advantages such as mild reaction condition and high selectivity which is the most promising process to realize industrialization.

The process includes two key technologies: oxalate synthesis and hydrogenation of oxalate. For the oxalate production technology, the oxalate is generated though a circulate catalytic process composed of coupling and regeneration with the attending of nitrite and CO, produced from coal gasification and the following separation process of pressure swing adsorption (PSA). DMO production process is a cycle system which has a mild reaction condition, good stability, high selectivity and low pollution. For the ethylene glycol production process: the produced oxalate is catalysis hydrogenation to ethylene glycol with hydrogen produced via PSA process. This process is a complex reaction system, mainly including the following reactions:

ROOCCOOR+2H₂→ROOCCH₂OH+ROH

ROOCCH₂OH+2H₂→HOCH₂CH₂OH+ROH

HOCH₂CH₂OH+H₂→C₂H₅OH+H₂O

The process on study mainly uses noble mental such as ruthenium as catalyst in liquid phase and copper as catalyst in gaseous phase. Because of the high pressure and the difficulty of the catalyst separation from liquid phase, people are concentrating on hydrogenation in gaseous phase on copper catalyst. CuCr was used as the catalyst which run for 460 h and the conversion of diethyl oxalate reached 100%, selectivity of ethylene glycol exceeded 95%. But because of Cr has great harm on human body and environmental impact, research of Cr-free catalyst gradually become the trend of catalyst for hydrogenation of oxalic ester. CuMoₖBaₚOₓ, was used as the catalyst using mixed ball milling process in 1985. Conversion of diethyl oxalate was 100% and yield of ethylene glycol reached as high as 97.7% on this catalyst. Ube reported a Cu/SiO₂ catalyst in 1986. With silica sol and copper ammonia solution as precursors and heated in the mixing state after mixing evenly to remove most of the water, resulted in the precipitation of copper and silicon oxide. After washing, drying and calcination, we prepared the catalyst. Conversion of diethyl oxalate was 100% and selectivity of ethylene glycol was 97.2% on the catalyst under reaction conditions of pressure 3 MPa, temperature 215° C., hydrogen ester ratio 30. American UCC has done a lot of research on copper-based chromium-free catalysts. They inspected different supporters (Al₂O₃, SiO₂, La₂O₃ etc), additives (Ag, Mo, Ba etc.) and preparation methods on the effect of catalytic reactivity and selectivity. Yield of ethylene glycol was up to 95% and time on stream was 455 h on the catalyst. UCC regarded the annular catalyst with a central hole as the ideal catalyst for oxalic ester hydrogenation to ethylene glycol. The special structure had better diffusion characteristics which can reduce local overheat. For mesoporous zeolite supported copper catalysts modified with additives such as magnesium, manganese, chromium, or aluminum in patent, conversion of oxalic ester reached 100% and selectivity of ethylene glycol achieved 96% on the catalyst under reaction conditions of temperature 210° C., pressure 3 MPa, hydrogen ester ratio 180, space velocity 0.5 h⁻¹. For an alumina supported copper catalyst modified with additives such as zinc, manganese, magnesium and chromium, with reaction pressure 0.3-1 MPa, temperature 145-200° C., mass space velocity of oxalate ester under 0.1-0.6 h⁻¹, conversion of oxalic ester was higher than 99% and selectivity of ethylene glycol achieved above 90%.

Researchers have achieved particular progress on catalyst used for hydrogenation of oxalic ester to ethylene glycol, but some problems still exist in further engineering scale up. Larger ratio of height to diameter of the catalyst bed will benefit the uniform distribution and easy control of the bed temperature, since the hydrogenation of oxalic ester to ethylene glycol is a temperature sensitive reaction. However, the increase in the height to diameter ratio will result in the increase of bed resistance. Additionally, the increase in particle size of the catalyst will easily cause local-pot overheat and enhanced side reactions. These factors severely restrict the industrialization process of hydrogenation of oxalic ester to ethylene glycol technology.

### SUMMARY OF THE INVENTION

In view of the above-described problems, it is one objective of the invention to provide a monolithic structure catalyst for producing ethylene glycol by hydrogenation of an oxalate. Active components of copper dispersed uniformly in the catalyst coating and the coating with thin layer form is evenly attached to the pore surface of cordierite or metal honeycomb with monolithic structure. It effectively reduces the resistance of internal diffusion and improves the activity and selectivity in the hydrogenation of oxalic ester to ethylene glycol.

It is another objective of the invention to provide a method for preparing a monolithic structure catalyst for producing ethylene glycol by hydrogenation of an oxalate. The method comprises following steps: first copper based catalyst powder is prepared by precipitation method, from which a catalyst slurry is further acquired, and then coated the catalyst slurry directly on the surface of the cordierite or metal honeycomb support to form the copper supported monolithic structure catalyst. The method ensures high dispersion of active component copper in the catalyst coating and enhances catalytic activity and thermal stability.

It is still another objective of the invention to provide a method for producing ethylene glycol by hydrogenation of an oxalate using a monolithic structure catalyst. The monolithic catalyst is used for hydrogenation of oxalic ester to ethylene glycol instead of particle catalyst, as it can reduce the cost by greatly reducing the pressure drop of the catalyst bed and decreasing the depletion of the catalyst, resulting from the abrasion during packing and reaction process. The provided monolithic catalyst has high catalytic activity, low resistance, convenient and quick replacement, which will benefit the realization of large-scale engineering amplification.

To achieve the above objectives, in accordance with one embodiment of the invention, there is provided a monolithic structure catalyst for producing ethylene glycol by hydrogenation of an oxalate, the catalyst comprising a carrier, an additive, and an active component, the carrier being ceramic or metallic honeycomb, the additive being Al, Si, Ba, Ca, Ti, Zr, Fe, Zn, Mn, V, La, Ce, an oxide thereof, or a mixture thereof, the active component being copper, and the active component and the additive being coated on the carrier to form a coating layer.

The additive accounts for 5-90 wt. % of the carrier, the active component accounts for 1-40 wt. % of the carrier, and the copper accounts for 5-50 wt. % of the coating layer.

The catalyst is prepared according to the following steps:
a) dissolving a soluble copper precursor with water to yield a solution A;
b) employing a soluble carbonate, bicarbonate, alkalis hydroxide, ammonia, urea, or a mixture thereof as a precipitant and mixing with the solution A;
c) adding an additive precursor into the solution A, stirring for 2-12 hours, heating to 50-120° C. for precipitating the copper and additive, stopping heating when a pH of the solution is less than 7, filtering, washing, drying, and calcinating a resulting precipitate to obtain a catalyst powder B;
d) squeezing, granulating, and sieving part of the catalyst powder B to form a granular catalyst C having 10-200 meshes, mechanically mixing another part of catalyst powder B, the granular catalyst C, an adhesive, and water to form a catalyst slurry D;
e) coating the catalyst slurry D onto the ceramic or metallic honeycomb using a dip coating method, drying, and calcinating to form a monolithic catalyst E; and
f) repeating step e) until a preset load is achieved

In a class of this embodiment, the additive is Al, Si, Ba, Ca, Ti, Zr, Fe, Zn, Mn, V, La, Ce, an oxide thereof, or a mixture thereof, and accounts for 10-50 wt. % of the carrier.

In a class of this embodiment, the copper is a main active component and accounts for 1-25 wt. % of the carrier.

In a class of this embodiment, the active component copper and the additive are coated on the honeycomb carrier in the form of the coating layer.

In a class of this embodiment, the coating layer comprising the active component and the additive accounts for 10-50 wt. % of the carrier, and the copper accounts for 10-40 wt. % of the coating layer.

In a class of this embodiment, the number of cells on the honeycomb is 50-1200 cells per square inch.

In accordance with another embodiment of the invention, there provided is a method for preparing a monolithic structure catalyst for producing ethylene glycol by hydrogenation of an oxalate, the method comprising the steps of:
a) dissolving a soluble copper precursor with water to yield a 0.2-2 M solution A;
b) employing a soluble carbonate, bicarbonate, alkalis hydroxide, ammonia, urea, or a mixture thereof as a precipitant and mixing with the solution A;
c) adding an additive precursor into the solution A, stirring for 2-12 hours, heating to 50-120° C. for precipitating the copper and additive, stopping heating when a pH of the solution is less than 7, filtering, washing, drying, and calcinating a resulting precipitate to obtain a catalyst powder B;
d) squeezing, granulating, and sieving part of the catalyst powder B to form a granular catalyst C having 10-200 meshes, mechanically mixing another part of catalyst powder B, the granular catalyst C, an adhesive, and water to form a catalyst slurry D, a mixing time being 0.5-24 hours, a rotating speed is 50-600 rpm, a mass ratio of the catalyst B, the granular catalyst C, the adhesive, and water is 0.02-0.6:0.02-0.8:0.03-0.2:1;
e) coating the catalyst slurry D onto the ceramic or metallic honeycomb using a dip coating method, drying at 60-140° C. for 2-24 hours, and calcinating at 200-600° C. for 1-10 hours to form a monolithic catalyst E; and
f) repeating step e) until a preset load is achieved.

In a class of this embodiment, the copper precursor is a nitrate, chloride, or acetate of copper.

In a class of this embodiment, the copper precursor is Cu(NO₃)₂.5H₂O.

In a class of this embodiment, the adhesive is selected from the group consisting of an inorganic adhesive water glass, silica sol, alumina, silica gel powder, an organic adhesive polyethylene glycol (PEG) 4000, PEG 5000, carboxymethylcellulose, sesbania powder, acetic acid, oxalic acid, and a mixture thereof.

In a class of this embodiment, the catalyst slurry D is prepared by mechanical agitation or ball-milling mixing, and the mass ratio of the catalyst B, the granular catalyst C, the adhesive, and water is 0.02-0.6:0.02-0.8:0.03-0.1:1.

The invention still provides a method for producing ethylene glycol by hydrogenation of an oxalate using a monolithic structure catalyst, the method comprising:
putting the monolithic structured catalyst into a fixed bed reactor, performing a reduction reaction in the presence of 5-20% H₂/N₂ at 250-450° C. for 2-20 hours, introducing pure hydrogen into the reactor, maintaining a reaction temperature at 190-260 ° C. and a reaction pressure at 1.0-5.0 MPa, vaporizing methanol solution comprising 10-25 wt. % dimethyl oxalate, liquid dimethyl oxalate, or diethyl oxalate in an evaporator, preheating, and introducing into the reactor, the liquid hourly space velocity (LHSV) of an oxalate being 0.2-1.5 g. mL⁻¹·h⁻¹, and a molar ratio of H₂/ester being 30-200.

In a class of this embodiment, the oxalate is dimethyl oxalate or diethyl oxalate.

In a class of this embodiment, the reaction temperature is 190-250° C.

In a class of this embodiment, the reaction pressure can be 2-4 MPa.

In a class of this embodiment, the molar ratio of H₂/ester is 50-200.

Advantages of the invention are summarized below:

The copper based monolithic catalyst in this invention is used in the hydrogenation of oxalate to ethylene glycol for the first time, which provides a new approach for the preparation of the catalyst used for the hydrogenation of oxalate to ethylene glycol.

Compared with granular catalysts, the monolithic catalyst in this invention shortens the internal diffusion path, improves the gas-solid mass transfer efficiency, and increases the effective contact area between reactant and catalyst. As a result, the catalyst increases the reactivity and selectivity of EG.

In the monolithic catalyst provided by this invention, the active component copper is dispersed uniformly in the coating layer supported on the honeycomb carrier. Thus, the catalyst has higher thermal stability.

The monolithic catalyst in this invention has lower bed resistant than granular catalyst, so it can work at the conditions of larger height-diameter ratio and high hydrogen-ester ratio. As a result, the catalytic performance and temperature distribution are improved, and the hot-spot temperature is decreased effectively.

Compared with granular catalyst, the monolithic catalyst in this invention has high conversion of oxalate and selectivity of EG in the hydrogenation of oxalate to ethylene glycol, and it doubles the LHSV which presents higher production capacity.

The monolithic catalyst in this invention runs for 500 hours stably in the hydrogenation of oxalate to ethylene glycol, average conversion of oxalate reaches 100% and selectivity of EG is higher than 96%, which presents high hydrogenation activity and stability.

Compared with granular catalysts, the monolithic catalyst in this invention is more convenient to be packed and replaced.

The monolithic catalyst in this invention contains no Cr and other toxic element, which is environmentally friendly.

### BRIEF DESCRIPTION OF ACCOMPANYING DRAWINGS

FIG. 1 shows an SEM image of a radial section of a monolithic catalyst; and

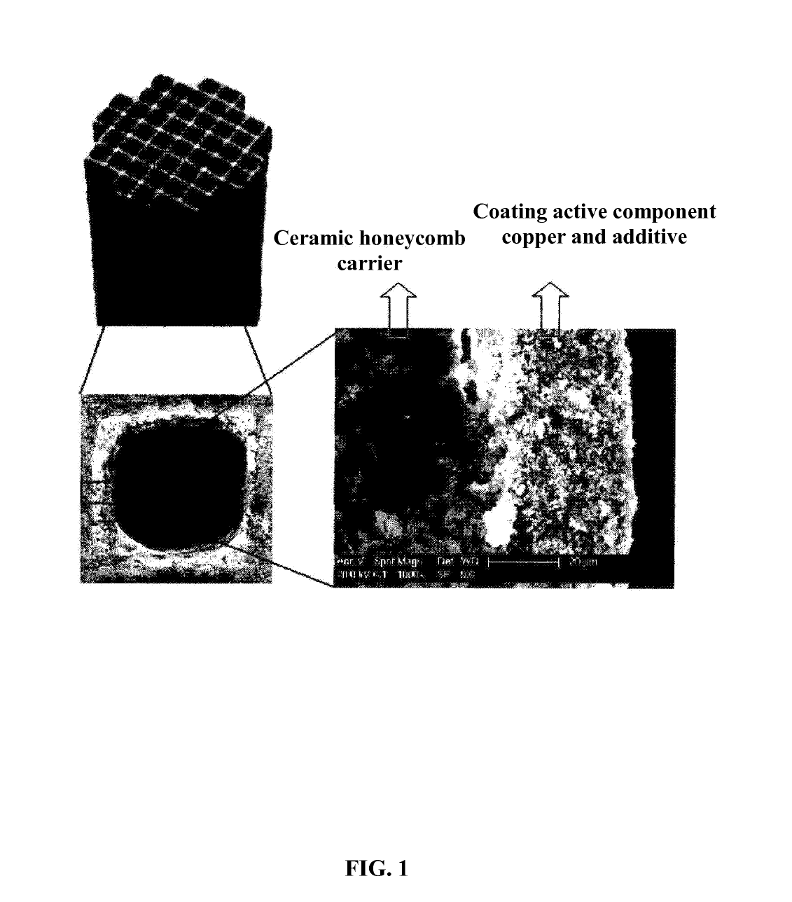

FIG. 2 shows stable data of a monolithic structure catalyst for producing ethylene glycol by hydrogenation of an oxalate.

### DETAILED DESCRIPTION OF THE EMBODIMENTS

For further illustrating the invention, experiments detailing a monolithic catalyst, a method for preparing and applying the same are described below. It should be noted that the following examples are intended to describe and not to limit the invention.

### EXAMPLE 1

Preparation of Monolithic Catalyst

15.5 g of Cu(NO₃)₂.5H₂O was dissolved in 150 mL water. 52 mL of 25 wt. % ammonia aqueous solution was added. Then 45 mL of 30 wt. % silica sol was added to the copper ammonia complex solution and aged by stirring for another 4 hours. The temperature was raised to 95° C. to allow for the precipitation of copper and silicate. The filtrate was washed with deionized water for 3 times, dried at 120° C. for 12 hours and calcined at 450° C. for 4 hours to form catalyst powder with Cu content of 20 wt. %.

Part of the catalyst powder was squeezed and sieved to 40-60 meshes. 8.0 g of squeezed catalyst, 8.0 g of catalyst powder, 0.5 g of pseudoboehmite and 50 mL of water was added in the ball mill can to ball mill at 200 rpm for 2 hours to get the catalyst slurry.

Cordierite carrier (Φ15×25 mm) of 400 cpsi was impregnated in the slurry for 5 min, then extra slurry on the carrier was blew off and dried at 120° C. for 12 hours, the coated cordierite carrier was then weighed. The above operation was repeated until the content of coat reached 20 wt. %. The as prepared catalyst was calcined at 450° C. for 6 hours to get the monolithic catalyst denoted as Cu/SiO₂/cordierite.

Catalytic Activity Test

The prepared monolithic catalyst was reduced by 20% H₂/N₂ with hydrogen flow rate of 50 mL/min in a fixed bed reactor at 350° C. for 4 hours. Pure hydrogen was introduced into the reactor, the temperature was controlled at 200° C. and the pressure was controlled at 2.4 MPa after reduction. 20 wt. % of dimethyl oxalate in methanol was introduced into the system by liquid high pressure pump. The LHSV of dimethyl oxalate is 0.8 h⁻¹, hydrogen-ester ratio is 90. Sample was analyzed by gas chromatography to calculate conversion and selection. Result is listed in Table 1.

TABLE 1
Catalytic performance of catalysts for producing ethylene glycol by
hydrogenation of an oxalate

| | Additive | Catalyst | C_{DMO}/% | S_{EG}/% |
|---|---|---|---|---|
| Example 1 | SiO₂ | Cu/SiO₂/cordierite | 99 | 96 |
| Example 2 | ZrO₂ | Cu/ZrO₂/cordierite | 88 | 80 |
| Example 3 | SiO₂—ZrO₂ | Cu/ZrO₂—SiO₂/cordierite | 100 | 97 |
| Example 4 | MnOₓ—SiO₂ | Cu/MnOₓ—SiO₂/cordierite | 98 | 88 |
| Example 5 | Al₂O₃—SiO₂ | Cu/Al₂O₃—SiO₂/cordierite | 99 | 94 |
| Example 6 | La₂O₃—SiO₂ | Cu/La₂O₃—SiO₂/cordierite | 98 | 92 |
| Example 7 | ZnO—SiO₂ | Cu/ZnO—SiO₂/cordierite | 95 | 85 |

### EXAMPLE 2

30 g of Zr(NO₃)₄.5H₂O was dissolved in 100 mL of 70% nitric acid in a 200 mL beaker and the concentration of Zr(NO₃)₄ was adjusted to 2 M by adding deionized water. Ammonia aqueous solution was then added to the above solution until the pH reached 4.0-5.0. Semitransparent zirconium sol formed and its concentration was adjusted to 1 M by adding deionized water. The above zirconium sol was aged for 24-48 hours under stirring.

The preparation method was the same as example 1, except that the silica sol was replaced by 127 mL of zirconium sol to make the catalyst slurry whose copper content was 20 wt. % and ZrO₂ content is 80%, the obtained monolithic catalyst was denoted as Cu/ZrO₂/cordierite whose coat content was 20 wt. %.

The catalyst was tested by the same method as Example 1, except that LHSV of oxalate was 0.6 h⁻¹, and the result was listed in Table 1.

### EXAMPLE 3

15.25 g of Cu(NO₃)₂.5H₂O was dissolved in 120 mL deionized water. Then 54.6 g of NaHCO₃ was added slowly to the solution. 39 mL of 30 wt. % silica sol and 16 mL of zirconium sol prepared in Example 2 was added to the above solution by drop and aged for 4 hours under stirring. The temperature was raised to 95° C. to allow for the precipitation of copper, silica and zirconia. Filtered and washed for 3 times. Dried at 120° C. for 12 hours and calcined at 450° C. for 4 hours to form the catalyst with 20 wt. % copper and 10 wt. % ZrO₂, which is denoted as Cu/ZrO₂—SiO₂.

Part of the above catalyst was squeezed and sieved to 40-60 meshes. 15 g of squeezed catalyst and 1.0 g of catalyst powder, 0.5 g of pseudoboehmite and 50 mL of water was added in the ball mill can to ball mill at 200 rpm for 2 hours to get the catalyst slurry.

Cordierite carrier (Φ15×25 mm) of 400 cpsi was impregnated in the slurry for 5 min, then extra slurry on the carrier was blew off and dried at 120° C. for 12 hours, the coated cordierite carrier was then weighed. The above operation was repeated until the content of coat reached 20 wt. %. The as prepared catalyst was calcined at 450° C. for 4 hours to get the monolithic catalyst denoted as Cu/ZrO₂—SiO₂/cordierite.

The catalyst was tested by the same method as Example 1, except that LHSV of oxalate was 1.2 h⁻¹, and the result was listed in Table 1.

### EXAMPLE 4

30.5 g of Cu(NO₃)₂.5H₂O was dissolved in 300 mL water. 104 mL of 25 wt. % ammonia aqueous solution was added. Then 78 mL of 30 wt. % silica sol and 30 mL of 2M manganese nitrate was added to the copper ammonia complex solution and aged by stirring for another 4 h. The temperature was raised to 80° C. to allow for the precipitation of copper, manganese and silicate. The filtrate was washed with deionized water for 3 times, dried at 120° C. for 12 hours and calcined at 450° C. for 4 hours to form catalyst powder with Cu content of 20 wt. % and manganese oxide content of 20 wt. %, which is denoted as Cu/MnOₓ—SiO₂.

Part of the above catalyst was squeezed and sieved to 80-100 meshes. 20.0 g of bead catalyst and 1.0 g of catalyst powder, 4 g of 30% silica sol and 50 mL of water was added in the ball mill can to ball mill at 200 rpm for 2 hours to get the slurry.

Cordierite carrier (Φ15×25 mm) of 400 cpsi was impregnating in the slurry for 5 min, then extra slurry on the carrier was blew off and dried at 80° C. for 12 hours, the coated cordierite carrier was then weighed. The above operation was repeated until the content of coat reached 20 wt. %. The as prepared catalyst was finally calcined at 400° C. for 4 hours to get the monolithic catalyst denoted as Cu/MnOₓ—SiO₂/cordierite.

The catalyst was tested by the same method as Example 1, except that reaction temperature was 200° C., and the result was listed in Table 1.

### EXAMPLE 5

Preparation of Monolithic Catalyst

30 g of Al(NO₃)₃.9H₂O was dissolved in 100 mL of 70% nitric acid in a 200 mL beaker and the concentration of Al(NO₃)₃ was adjusted to 2M by adding deionized water. Ammonia aqueous solution was then added to the above solution until the Al(NO₃)₃ was totally precipitated. Diluted nitric acid was added until the precipitate was dissolved. 1M of semitransparent aluminum sol formed after stirring for 3-4 hours and aging overnight.

15.25 g of Cu(NO₃)₂.5H₂O was dissolved in 120 mL deionized water. Then 54.6 g of NaHCO₃ was added slowly to the solution. 39 mL of 30 wt. % silica sol and 41 mL of as prepared aluminum sol was added to the above solution by drop and aged for 10 hours under stirring. The temperature was raised to 70° C. to allow for the precipitation of copper, silica and alumina. The filtrate was washed for 3 times and dried at 120° C. for 12 hours and finally calcined at 450° C. for 4 hours to form the catalyst, whose copper content is 20 wt. % and Al₂O₃ content is 10 wt. %, which is denoted as Cu/Al₂O₃—SiO₂.

Part of the catalyst powder was squeezed and sieved to 80-100 meshes. The 6.0 g of squeezed catalyst, 12.0 g of catalyst powder, 2 g of silica powder and 50 mL of water was added in the ball mill can to ball mill at 200 rpm for 2 hours to get the catalyst slurry.

Cordierite carrier (Φ15×25 mm) of 400 cpsi was impregnating in the slurry for 3 min, then extra slurry on the carrier was blew off and dried at 120° C. for 8 hours, the coated cordierite carrier was then weighed. The above operation was repeated until the content of coat reached 20 wt. %. The as prepared catalyst was calcined at 450° C. for 4 hours to get the monolithic catalyst denoted as Cu/Al₂O₃—SiO₂/cordierite.

Catalytic Activity Test

The as prepared monolithic catalyst was reduced by 10% H₂/N₂ with hydrogen flow rate of 100 mL/min in the fixed bed reactor at 400° C. for 10 hours. Pure hydrogen was introduced into the reactor, the temperature was controlled at 200° C. and the pressure was controlled at 4 MPa after reduction. 20 wt. % of dimethyl oxalate in methanol was introduced into the system by liquid high pressure pump. The LHSV of dimethyl oxalate is 0.8 h⁻¹, hydrogen-ester ratio is 70. Sample was analyzed by gas chromatography to calculate conversion and selection. Result is listed in Table 1.

### EXAMPLE 6

Preparation of Monolithic Catalyst

30.5 g of copper nitrate was dissolved in 150 mL water. 104 mL of 25 wt. % ammonia aqueous solution was added. Then 78 mL of 30 wt. % silica sol and 7.0 mL of 2M lanthanum nitrate was added to the copper ammonia complex solution and aged by stirring for another 4 hours. The temperature was raised to 80° C. to allow for the precipitation of copper, lanthanum and silicate. The filtrate was washed with deionized water for 3 times, dried at 120° C. for 12 hours and calcined at 450° C. for 4 hours to form catalyst powder with Cu content of 20 wt. % and La₂O₃ content of 10 wt. % which is denoted as Cu/La₂O₃—SiO₂.

Part of the catalyst powder was squeezed and sieved to 180-200 meshes. The 6.0 g of bead catalyst, 12.0 g of catalyst powder, 2 g of silica powder and 50 mL of water was added in the ball mill can to ball mill at 200 rpm for 2 hours to get the slurry.

Cordierite carrier (Φ15×25 mm) of 400 cpsi was impregnating in the slurry for 3 min, then extra slurry on the carrier was blew off and dried at 120° C. for 8 hours, the coated cordierite carrier was then weighed. The above operation was repeated until the content of coat reached 20 wt. %. The as prepared catalyst was calcined at 450° C. for 4 hours to get the monolithic catalyst denoted as Cu/La₂O₃—SiO₂/cordierite.

Catalytic Activity Test

The as prepared monolithic catalyst was reduced by 20% H₂/N₂ with hydrogen flow rate of 50 mL/min in the fixed bed reactor at 300° C. for 20 hours. Pure hydrogen was introduced into the reactor, the temperature was controlled at 190° C. and the pressure was controlled at 3 MPa after reduction. 20 wt. % of dimethyl oxalate in methanol was introduced into the system by liquid high pressure pump. The LHSV of dimethyl oxalate is 1.0 h⁻¹, hydrogen-ester ratio is 90. Sample was analyzed by gas chromatography to calculate conversion and selection. Result is listed in Table 1.

### EXAMPLE 7

The preparation method was the same as Example 4, except that the manganese nitrate was replaced by 13 mL solution of Zinc nitrate (2M) to make the catalyst powder Cu/ZnO—SiO₂, which was used to prepare the catalyst slurry whose copper content was 20 wt. % and ZnO content is 10%. The obtained monolithic catalyst was denoted as Cu/ZnO—SiO₂/cordierite whose coat content was 20 wt. %. The catalyst was tested by the same method as Example 4, and the result was listed in Table 1.

### EXAMPLES 8-10

The preparation and test method was the same as Example 1, except that the dip coating times was changed to obtain monolithic catalysts with different coating content of 10%, 30% and 40% based on the honeycomb carrier, whose corresponding copper content were 2%, 6% and 8% respectively. Results were listed in Table 2.

TABLE 2
Catalytic performance of catalyst Cu/SiO₂/cordierite with different
coating and copper loads

| | Coat | Cu | | | |
|---|---|---|---|---|---|
| | content | content | | | |
| | wt. % | wt. % | Catalyst | C_{DMO}/% | S_{EG}/% |
| Example 8 | 10 | 2 | Cu/SiO₂/cordierite | 80 | 85 |
| Example 9 | 30 | 6 | Cu/SiO₂/cordierite | 100 | 95 |
| Example 10 | 40 | 8 | Cu/SiO₂/cordierite | 100 | 92 |
| Example 11 | 50 | 20 | Cu/SiO₂/cordierite | 100 | 93 |

### EXAMPLE 11

Preparation of Monolithic Catalyst

31.0 g of Cu(NO₃)₂.5H₂O was dissolved in 300 mL deionized water. Then 25.5 g of urea was added slowly to the solution. 45 mL of 30 wt. % silica sol was added to the above solution by drop and aged for 4 hours under stirring. The temperature was raised to 95° C. to allow for the precipitation of copper, silica. Filter was washed for 3 times and dried at 120° C. for 12 hours and calcined finally at 450° C. for 4 hours to form the catalyst with copper content of 40 wt. % and SiO₂ content of 60 wt. %.

Part of the catalyst powder was squeezed and sieved to 180-200 meshes. The 1.0 g of squeezed catalyst, 23.0 g of catalyst powder, 0.3 g of PEG4000, 0.3 g of Sesbania powder and 50 mL of water was mixed together by mechanical stirring to get the catalyst slurry.

Cordierite carrier (Φ15×25 mm) of 400 cpsi was impregnated in the slurry for 3 min, then extra slurry on the carrier was blew off and dried at 120° C. for 20 hours, the coated cordierite carrier was then weighed. The above operation was repeated until the content of coat reached 50 wt. % whose copper content was 20 wt. %. The as prepared catalyst was calcined at 450° C. for 6 hours to get the monolithic catalyst denoted as Cu/SiO₂/cordierite.

Catalytic Activity Test

The as prepared monolithic catalyst was reduced by 20% H₂/N₂ with hydrogen flow rate of 50 mL/min in the fixed bed reactor at 250° C. for 10 hours. Pure hydrogen was introduced into the reactor, the temperature was controlled at 200° C. and the pressure was controlled at 3 MPa after reduction. 20 wt. % of dimethyl oxalate in methanol was introduced into the system by liquid high pressure pump. The LHSV of dimethyl oxalate is 1.2 h⁻¹, hydrogen-ester ratio is 90. Sample was analyzed by gas chromatography to calculate conversion and selection. Result is listed in Table 2.

### EXAMPLES 12-17

The preparation and test method was the same as Example 1, except that the milling time was changed to 0.5, 5 or 10 hours, or the rotation speed was changed to 100, 300 or 500 rpm respectively. Results were listed in Table 3.

TABLE 3
Catalytic performance of Cu/SiO₂/cordierite prepared with different milling
time and rotational speed

| | Milling | Rotation | | | |
|---|---|---|---|---|---|
| | time/h | speed/rpm | Catalyst | C_{DMO}/% | S_{EG}/% |
| Example 12 | 0.5 | 200 | Cu/SiO₂/cordierite | 98 | 91 |
| Example 13 | 5 | 200 | Cu/SiO₂/cordierite | 99 | 93 |
| Example 14 | 10 | 200 | Cu/SiO₂/cordierite | 94 | 86 |
| Example 15 | 2 | 100 | Cu/SiO₂/cordierite | 99 | 93 |
| Example 16 | 2 | 300 | Cu/SiO₂/cordierite | 100 | 94 |
| Example 17 | 2 | 500 | Cu/SiO₂/cordierite | 95 | 90 |

### EXAMPLE 18

Preparation of Monolithic Catalyst

The catalyst powder B is prepared by the same method as catalyst Cu/ZrO₂/SiO₂ mentioned in Example 3. The catalyst slurry is obtained by adding 1.0 g catalyst Cu/ZrO₂/SiO₂, 15 g extruded catalyst (mesh 40-60), 0.5 g pseudoboehmite, 0.5 g oxalic acid and 50 mL water into the grinding mill and ball-milling for 4 hours at 250 rpm.

The cordierite support (400 cpsi, Φ15×25 mm) was impregnated in the slurry for 5 min and blow off the surplus slurry. Then the support was dried for 8 hours at 120° C. and weighed. Repeat the above steps until the weight of the coating is above 20% of the support. The Cu/ZrO₂—SiO₂/cordierite monolithic catalyst is finally obtained after 4 hours calcination at 450° C.

The activity test was performed by the same method as Example 1 except that that the LHSV of DMO was 1.0 h⁻¹. The resulting C_{DMO} is 100% and S_{EG} is 96%.

### EXAMPLES 19-20

The preparation and test method of catalyst is the same as Example 1 except that the support was calcined for 6 hours at 350° C. and 550° C. separately. The results of activity test are listed in Table 4.

TABLE 4
Performance of catalysts prepared with different calcination temperatures

| | Calcination | | | |
|---|---|---|---|---|
| | Temperature/° C. | Catalyst | C_{DMO}/% | S_{EG}/% |
| Example 19 | 350 | Cu/SiO₂/cordierite | 94 | 91 |
| Example 20 | 550 | Cu/SiO₂/cordierite | 86 | 81 |

### EXAMPLES 21-23

The preparation and test method of catalyst is the same as Example 1 except that calcination time is 1, 8 or 10 hours at 450° C. The result of activity test is listed in Table 5.

TABLE 5
Performance of catalysts prepared with different calcination time

| | Calcination time/h | Catalyst component | C_{DMO}/% | S_{EG}/% |
|---|---|---|---|---|
| Example 21 | 1 | Cu/SiO₂/cordierite | 83 | 78 |
| Example 22 | 8 | Cu/SiO₂/cordierite | 98 | 92 |
| Example 23 | 10 | Cu/SiO₂/cordierite | 87 | 82 |

### EXAMPLE 24

The preparation and test method of catalyst is the same as Example 1 except that the support used is 900 cpsi. The result of activity test is listed in Table 6.

TABLE 6
Performance of catalysts prepared with different honeycomb
specifications

| | Honeycomb | Catalyst | | |
|---|---|---|---|---|
| | specification/cpsi | component | C_{DMO}/% | S_{EG}/% |
| Example 24 | 900 | Cu/SiO₂/cordierite | 94 | 88 |
| Example 25 | 200 | Cu/SiO₂/cordierite | 92 | 87 |

### EXAMPLE 25

The preparation and test method of catalyst is the same as Example 1 except that the support used is 200 cpsi. The result of activity test is listed in Table 6.

### EXAMPLE 26

After high temperature treatment of 10 hours, the corrugated sheets are impregnated in the slurry (mentioned in Example 1) and calcined for 6 hours at 450° C. Roll the sheets to honeycomb shape and the monolithic catalyst Cu/SiO₂/Metal is obtained. The slurry weight is 20% of the honeycomb support and the Cu loading is 4%. The activity test is carried out by the same method as Example 1. The result is that C_{DMO} is 97% and S_{EG} is 91%.

### EXAMPLE 27

The implementary conditions are the same as Example 1 except that the DMO is replaced by DEO as reactant. The activity test result turns to be that C_{DMO} is 96% and S_{EG} is 91%.

### EXAMPLE 28

The monolithic catalyst prepared in Example 1 is placed in the fix-bed reactor and reduced by 20% H₂/N₂ under the following conditions: hydrogen flow rate 50 mL/min, temperature 250° C. for 20 h. After reduction, the system is filled with pure hydrogen and controlled under the following conditions: temperature 250° C. and pressure 2.0 MPa. 20 wt. % DMO in methanol is pumped into the system. The LHSV of DMO is controlled at 1.5⁻¹ and the ratio of H₂/DMO is 50. C_{DMO} and S_{EG} are calculated from timing analysis results of production components with GC. The result is listed in Table 7.

TABLE 7
Performance of catalysts at different H₂/DMO

| | Ratio of H₂/DMO | C_{DMO}/% | S_{EG}/% |
|---|---|---|---|
| Example 28 | 50 | 100 | 93 |
| Example 29 | 150 | 100 | 96 |
| Example 30 | 200 | 100 | 91 |

### EXAMPLE 29

The implementary conditions are the same as Example 1 except that the ratio of H₂/DMO is 150. The result is listed in Table 7.

### EXAMPLE 30

The implementary conditions are the same as Example 1 except that the ratio of H₂/DMO is 200. The result is listed in Table 7.

### EXAMPLE 31

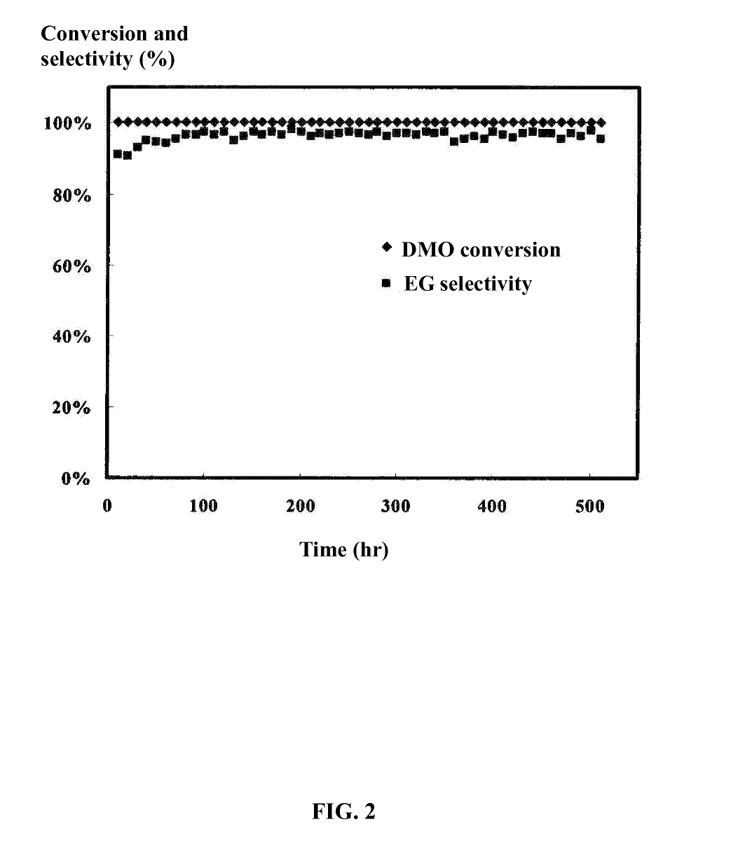

Other conditions are the same as Example 3 except that the activity of catalyst is tested under the following conditions: LHSV of DMO 0.8 h⁻¹, the ratio of H₂/DMO 60-90, temperature 200-210° C., pressure 2.0-3.0 MPa. The stability test lasts for 500 h. Average C_{DMO} is 100% and average S_{EG} is 96%. No evident decline in catalytic activity was found during the test. The details are shown in FIG. 2.

### COMPARATIVE EXAMPLE 1

15.5 g of Cu(NO₃)₂.5H₂O was dissolved in 150 mL water. 52 mL of 25 wt. % ammonia aqueous solution was added. Then 45 mL of 30 wt. % silica sol was added to the copper ammonia complex solution and aged by stirring for another 4 hours. The temperature was raised to 95° C. to allow for the precipitation of copper and silica. The filtrate was washed with deionized water for 3 times, dried at 120° C. for 12 hours and calcined at 450° C. for 4 hours to form catalyst powder with Cu content of 20 wt. %.

The above Cu/SiO2 catalyst powder is extruded to Φ5×4 mm particles. 2.00 mL as prepared catalyst is reduced by 20% H₂/N₂ with hydrogen flow rate of 50 mL/min in the fixed bed reactor at 350° C. for 4 hours. After reduction, the system is filled with pure hydrogen and controlled under the following conditions: temperature 200° C. and pressure 2.5 MPa. 20 wt. % DMO in methanol is pumped into the system. The LHSV of DMO is controlled at 0.4 h⁻¹ and the ratio of H₂/DMO is 90. C_{DMO} and S_{EG} are calculated from timing analysis results of production components with GC. The result is that C_{DMO} is 98% and S_{EG} is 90%.

The monolithic catalyst provided by the invention is applied in the synthesis process of EG via hydrogenation of DMO. Compared with supported granular catalyst (Comparative Example 1), the monolithic catalyst shows a better performance in C_{DMO} and S_{EG}. Furthermore, the technology using monolithic catalyst can get a larger production of EG due to that LHSV of DMO and STY of EG are twice the conventional supported catalyst.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A catalyst, comprising:
1) a carrier, said carrier being ceramic or metallic honeycomb;
2) an additive, said additive being Al, Si, Ba, Ca, Ti, Zr, Fe, Zn, Mn, V, La, Ce, an oxide thereof, or a mixture thereof; and
3) an active component, said active component being copper, and said active component and said additive being coated on said carrier to form a coating layer;
wherein
said additive accounts for 5-90 wt. % of said carrier, said active component accounts for 1-40 wt. % of said carrier, and said copper accounts for 5-50 wt. % of said coating layer; and
said catalyst is prepared according to the following steps:
a) dissolving a soluble copper precursor with water to yield a solution A;
b) employing a soluble carbonate, bicarbonate, alkalis hydroxide, ammonia, urea, or a mixture thereof as a precipitant and mixing with said solution A;
c) adding an additive precursor into said solution A, stirring for 2-12 hours, heating to 50-120° C. for precipitating said copper and additive, stopping heating when a pH of said solution is less than 7, filtering, washing, drying, and calcinating a resulting precipitate to obtain a catalyst powder B;
d) squeezing, granulating, and sieving part of said catalyst powder B to form a granular catalyst C having 10-200 meshes, mechanically mixing another part of catalyst powder B, said granular catalyst C, an adhesive, and water to form a catalyst slurry D;
e) coating said catalyst slurry D onto said ceramic or metallic honeycomb using a dip coating method, drying, and calcinating to form a monolithic catalyst E; and
f) repeating step e) until a preset load is achieved.

2. The catalyst of claim 1, wherein said additive accounts for 10-45 wt. % of said carrier.

3. The catalyst of claim 1, wherein said additive is Al, Si, Zr, Zn, Mn, La, an oxide thereof, or a mixture thereof.

4. The catalyst of claim 1, wherein said copper is a main active component and accounts for 1-25 wt. % of said carrier.

5. The catalyst of claim 1, wherein said coating layer comprising said active component and said additive accounts for 10-50 wt. % of said carrier, and said copper accounts for 10-40 wt. % of said coating layer.

6. The catalyst of claim 1, wherein the number of cells on said honeycomb is 50-1200 cells per square inch.

7. A method for preparing a catalyst, the method comprising the steps of:
a) dissolving a soluble copper precursor with water to yield a 0.2-2 M solution A;
b) employing a soluble carbonate, bicarbonate, alkalis hydroxide, ammonia, urea, or a mixture thereof as a precipitant and mixing with said solution A;
c) adding an additive precursor into said solution A, stirring for 2-12 hours, heating to 50-120° C. for precipitating said copper and additive, stopping heating when a pH of said solution is less than 7, filtering, washing, drying, and calcinating a resulting precipitate to obtain a catalyst powder B;
d) squeezing, granulating, and sieving part of said catalyst powder B to form a granular catalyst C having 10-200 meshes, mechanically mixing another part of catalyst powder B, said granular catalyst C, an adhesive, and water to form a catalyst slurry D, a mixing time being 0.5-24 hours, a rotating speed is 50-600 rpm, a mass ratio of said catalyst powder B, said granular catalyst C, said adhesive, and water is 0.02-0.6:0.02-0.8:0.03-0.2:1;
e) coating said catalyst slurry D onto said ceramic or metallic honeycomb using a dip coating method, drying at 60-140° C. for 2-24 hours, and calcinating at 200-600° C. for 1-10 hours to form a monolithic catalyst E; and
f) repeating step e) until a preset load is achieved

8. The method of claim 7, wherein said copper precursor is a nitrate, chloride, or acetate of copper.

9. The method of claim 7, wherein said copper precursor is Cu(NO₃)₂.5H₂O.

10. The method of claim 7, wherein said adhesive is selected from the group consisting of water glass, silica sol, alumina, silica gel powder, polyethylene glycol (PEG) 4000, PEG 5000, carboxymethylcellulose, sesbania powder, acetic acid, oxalic acid, and a mixture thereof.

11. The method of claim 7, wherein said catalyst slurry D is prepared by mechanical agitation or ball-milling mixing, and the mass ratio of said catalyst B, said granular catalyst C, said adhesive, and water is 0.01-0.6:0.01-0.8:0.03-0.1:1.

12. A method for producing ethylene glycol by hydrogenation of an oxalate using a catalyst of claim 1, the method comprising:
a) putting said catalyst into a fixed bed reactor;
b) performing a reduction reaction in the presence of 5-20% H₂/N₂ at 250-450° C. for 2-20 hours;
c) introducing pure hydrogen into said reactor, and maintaining a reaction temperature at 190-260 ° C. and a reaction pressure at 1.0-5.0 MPa;
d) vaporizing and preheating methanol solution comprising 10-25 wt. % dimethyl oxalate, liquid dimethyl oxalate, or diethyl oxalate in an evaporator; and
e) introducing said methanol solution, liquid dimethyl oxalate, or diethyl oxalate into said reactor, and controlling the liquid hourly space velocity (LHSV) of said oxalate at 0.2-1.5 g. mL⁻¹·h⁻¹, and a molar ratio of H₂/ester at 30-200.

13. The method of claim 12, wherein said oxalate is dimethyl oxalate or diethyl oxalate.

14. The method of claim 12, wherein said reaction temperature is 190-250° C.

15. The method of claim 12, wherein said reaction pressure is 2-4 MPa.

16. The method of claim 12, wherein said molar ratio of H₂/ester is 50-200.
